(19) United States
(12) Patent Application Publication
(10) Pub. No.: US 2021/0374950 A1
(43) Pub. Date: Dec. 2, 2021

(54) SYSTEMS AND METHODS FOR VESSEL PLAQUE ANALYSIS

(71) Applicant: SHENZHEN KEYA MEDICAL TECHNOLOGY CORPORATION, Shenzhen (CN)

(72) Inventors: Feng Gao, Seattle, WA (US); Zhenghan Fang, Shoreline, WA (US); Yue Pan, Seattle, WA (US); Junjie Bai, Seattle, WA (US); Youbing Yin, Kenmore, WA (US); Hao-Yu Yang, Seattle, WA (US); Kunlin Cao, Kenmore, WA (US); Qi Song, Seattle, WA (US)

(73) Assignee: SHENZHEN KEYA MEDICAL TECHNOLOGY CORPORATION, Shenzhen (CN)

(21) Appl. No.: 17/121,595

(22) Filed: Dec. 14, 2020

Related U.S. Application Data
(60) Provisional application No. 63/030,248, filed on May 26, 2020.

(51) Int. Cl. G06T 7/00; G06K 9/62; G06T 3/40; G06T 7/70; A61B 6/03; A61B 6/00

(52) U.S. Cl. CPC G06T 7/0012; G06T 2207/20081; G06K 9/6232; G06T 3/40; G06T 7/70; A61B 6/032; A61B 6/504; A61B 6/5217; G06K 2209/05; G06T 2207/30101; G06T 2207/20084; G06T 2207/10081; G06T 2207/10016; G06T 2200/04; G06T 2207/20076; G06K 9/6267

(57) ABSTRACT

The disclosure relates to systems and methods for vessel image analysis. The method includes receiving a set of images along a vessel acquired by a medical imaging device, and determining a sequence of centerline points along the vessel and a sequence of image patches at the respective centerline points based on the set of images. The method further includes detecting plaques based on the sequence of image patches using a first learning network. The first learning network includes an encoder configured to extract feature maps based on the sequence of image patches and a plaque range generator configured to generate a start position and an end position of each plaque based on the extracted feature maps. The method also includes classifying each detected plaque and determining a stenosis degree for the detected plaque, using a second learning network reusing at least part of the parameters of the first learning network and the extracted feature maps.

### CROSS REFERENCE TO RELATED APPLICATION

This application is based on and claims the priority of U.S. Provisional Application No. 63/030,248, filed on May 26, 2020, which is incorporated herein by reference in its entirety.

### TECHNICAL FIELD

The present disclosure relates to a device and system for medical image analysis, and more specifically, to a device and system for vessel plaque analysis based on medical images using a learning network.

### BACKGROUND

Vascular diseases have become a common threat human health. A considerable number of vascular diseases are caused by the accumulation of plaque on the vessel wall, but current detection, analysis and diagnosis of these plaques provide suboptimal results.

Using Coronary artery disease (CAD) as an example, which refers to the narrowing or blockage of the coronary arteries. It is the most common type of heart diseases and is usually caused by the buildup of atherosclerotic plaques in the wall of the coronary arteries. Patients with coronary arteries narrowed or occluded by plaques, i.e. stenosis, will suffer from limited blood supply to the myocardium and may have myocardial ischemia. Further, if the plaques rupture, the patient may develop acute coronary syndromes or even worse, a heart attack (myocardial infarction). According to the composition of an atherosclerotic plaque, it can be further classified as calcified plaque, non-calcified plaque, and mixed plaque (i.e., with both calcified and non-calcified components). The stability of a plaque varies based on its composition. A calcified plaque is relatively stable, while a non-calcified or mixed plaque is unstable and more likely to rupture.

However, non-calcified plaques or mixed plaques with a higher risk are more difficult to detect or more complicated to detect using existing medical imaging means. Coronary CT angiography (CCTA) is a commonly used non-invasive approach for the analysis of CADs and coronary artery plaques. Taking CCTA as an example, the detection of non-calcified and mixed plaques from a CCTA is more complicated. The plaques are easily missed or confused with surrounding tissues as the contrast of the plaques to surrounding tissues is much lower.

Atherosclerotic plaques are scattered on the vessel walls of complicated coronary arteries (for example, the anterior descending branch of the left coronary artery, the main trunk of the right coronary artery, the main trunk of the left coronary artery and the left circumflex artery) and present multiplicity. Therefore, the analysis and diagnosis of plaque is a difficult and time-consuming task. This is true even for experienced radiologists and cardiovascular specialists. For example, a comprehensive manual scan of the coronary arteries also results in heavy workload and work intensity. Radiologists and cardiovascular specialists may miss local plaques even in a comprehensive scan of the coronary arteries, especially may miss non-calcified plaques and mixed plaques (which are of high risk) with similar CT density to that of the surrounding tissues. Furthermore, even if a plaque is detected, the classification error of the plaque type will seriously affect the diagnosis results of radiologists and cardiovascular experts, resulting in subsequent overtreatment or undertreatment. The accuracy of classification of plaque types relies heavily on the experience of radiologists and cardiovascular experts, and differs obviously among individuals.

Although some vascular plaque analysis algorithms have recently been proposed trying to assist radiologists in daily diagnostic procedures and reduce their workload, these algorithms have the following disadvantages. Some of them require a lot of manual interactions (such as voxel-level annotation). Some require complicated and time-consuming auxiliary analysis in advance, such as vessel lumen segmentation, vessel health diameter estimation and vessel wall morphology analysis. Some can only provide local analysis for the vessel, and cannot satisfy clinical needs in terms of the level of automation, the complexity of the calculation (involving the detection phase and the training phase), operational convenience and user-friendliness. Therefore, there is still room for improving the prior vascular plaque analysis algorithms.

### SUMMARY

The present disclosure is provided to address the above-mentioned problems existing in the prior art. Systems and methods are disclosed for vessel plaque analysis, which can automatically and flexibly detect and locate plaque for any branch, path, segment of a vessel or the entire vascular tree accurately and quickly in an end-to-end manner, and determine the type and the degree of stenosis for each detected plaque. The disclosed systems and methods effectively reduce the computational complexity (involving the detection phase and the training phase), and significantly improve the operating convenience and user-friendliness.

According to a first aspect of the present disclosure, a method for vessel plaque analysis is provided. The method includes receiving a set of images along a vessel acquired by a medical imaging device, and determining a sequence of centerline points along the vessel and a sequence of image patches at the respective centerline points based on the set of images. The method further includes detecting plaques based on the sequence of image patches using a first learning network. The first learning network includes an encoder configured to extract feature maps based on the sequence of image patches and a plaque range generator configured to generate a start position and an end position of each plaque based on the extracted feature maps. The method also includes classifying each detected plaque and determining a stenosis degree for the detected plaque, using a second learning network reusing at least part of the parameters of the first learning network and the extracted feature maps.

According to a second aspect of the present disclosure, a system for vessel plaque analysis is provided. The system may include an interface and a processor. The interface may be configured to receive a set of images along a vessel acquired by a medical imaging device. The processor may be configured to reconstruct a 3D model of the vessel based on the set of images of the vessel, and extract a sequence of centerline points along the vessel and a sequence of image patches at the respective centerline points. The processor may be further configured to detect plaques based on feature maps extracted from the sequence of image patches and generate the start position and the end position of each plaque based on the extracted feature maps, using a first learning network. The processor is further configured to classify each detected plaque and determine the stenosis degree for each detected plaque, using a second learning network reusing at least part of the parameters of the first learning network and the extracted feature maps.

According to a third aspect of the present disclosure, a non-transitory computer-readable storage medium is provided with computer-executable instructions stored thereon. The computer-executable instructions, when executed by a processor, may perform the method for vessel plaque analysis. The method includes receiving a set of images along a vessel acquired by a medical imaging device, and determining a sequence of centerline points along the vessel and a sequence of image patches at the respective centerline points based on the set of images. The method further includes detecting plaques based on the sequence of image patches using a first learning network. The first learning network includes an encoder configured to extract feature maps based on the sequence of image patches and a plaque range generator configured to generate a start position and an end position of each plaque based on the extracted feature maps. The method also includes classifying each detected plaque and determining a stenosis degree for the detected plaque, using a second learning network reusing at least part of the parameters of the first learning network and the extracted feature maps.

The disclosed systems and methods for vessel plaque analysis according to various embodiments of the present disclosure may automatically and flexibly detect and locate a plaque for any branch, path, segment of a vessel or the entire vascular tree accurately and quickly in an end-to-end manner, and determine the type and the stenosis degree of each detected plaque. As a result, they effectively reduce the computational complexity (involving the detection phase and the training phase), and significantly improve the operating convenience and user-friendliness.

It should be understood that the foregoing general description and the following detailed description are only exemplary and illustrative, and do not intend to limit the claimed invention.

### BRIEF DESCRIPTION OF THE DRAWINGS

In the drawings that are not necessarily drawn to scale, similar reference numerals may describe similar components in different views. Similar reference numerals with letter suffixes or different letter suffixes may indicate different examples of similar components. The drawings generally show various embodiments by way of example and not limitation, and together with the description and claims, are used to explain the disclosed embodiments. Such embodiments are illustrative and are not intended to be exhaustive or exclusive embodiments of the method, system, or non-transitory computer-readable medium having instructions for implementing the method thereon.

FIG. 1 shows a schematic diagram of the configuration and working principle of a device for vessel plaque analysis according to an embodiment of the present disclosure.

FIG. 2 shows an exemplary diagram of 3D convolution performed by an encoder of a plaque detection unit in the device of FIG. 1, according to the embodiment of the present disclosure.

FIG. 3 shows an exemplary diagram of a plaque detection unit compatible with the device of FIG. 1, according to the embodiment of the present disclosure.

FIG. 4 shows an exemplary diagram of a learning network for vessel plaque analysis, according to the embodiment of the present disclosure.

FIG. 5 shows an exemplary diagram of another learning network for vessel plaque analysis, according to the embodiment of the present disclosure.

FIG. 6 shows an exemplary diagram of the encoder and decoder compatible with the learning network shown in FIG. 5, according to the embodiment of the present disclosure.

FIG. 7 shows a flowchart for an exemplary method training a learning network for vessel plaque analysis, according to the embodiment of the present disclosure.

FIG. 8 shows a flowchart an exemplary method for vessel plaque analysis using a learning network, according to the embodiment of the present disclosure.

FIG. 9 shows a block diagram of a system for vessel plaque analysis, according to an embodiment of the present disclosure.

### DETAILED DESCRIPTION

Reference will now be made in detail to exemplary embodiments, examples of which are illustrated in the drawings. In this disclosure, a vessel may include any one of coronary artery, carotid artery, abdominal aorta, cerebral vessel, ocular vessel, and femoral artery, etc. In some embodiments, a sequence of centerline points may be determined along the vessel. The centerline points may be determined from at least a part of the vessel in the sequence, e.g., a branch, segment, path, any part of the tree structure of the vessel, and or the whole vessel segment or the entire vessel tree, which is not specifically limited here. In the following particular embodiments, a series of centerline points of a vessel part along a single path are taken as an example for description, but the present disclosure is not limited to this. Instead, one may adjust the number and locations of the centerline points according to the vessel of interest (including part or whole) intended for plaque analysis. The structural framework and the number of nodes may also be adjusted accordingly. In some embodiments, the information propagation manner among nodes may be adjusted according to the spatial constraint relationship among the respective centerline points, so as to obtain a learning network adapted for the vessel of interest. According to some embodiments, image patches may be obtained at the respective centerline points. The image patches may spatially enclose the respective centerline points therein. For example, an image patch may be a 2D slice image relative to the center line at the centerline point, or a 3D volume image patch around the centerline point.

In the description of the learning network herein, for the convenience of description, the description of the activation layer is omitted. For example, in some embodiments, after the convolutional layer (usually before the corresponding pooling layer) there may be an activation function layer (such as the RELU function layer). For another example, the output of the neuron in the fully connected layer can be provided with an activation function layer (such as a Sigmoid function layer), which is not specifically shown but contemplated here. The present disclosure uses expressions “first”, “second”, “third”, “fourth”, “fifth”, “sixth” and “seventh” only to distinguish the described components rather than to implying any limitation on numbers of such components. In describing the methods, it is contemplated that the various steps is not necessarily executed in the exact order shown in the drawings. The steps can be performed in any technically feasible order different from the order shown in the drawings.

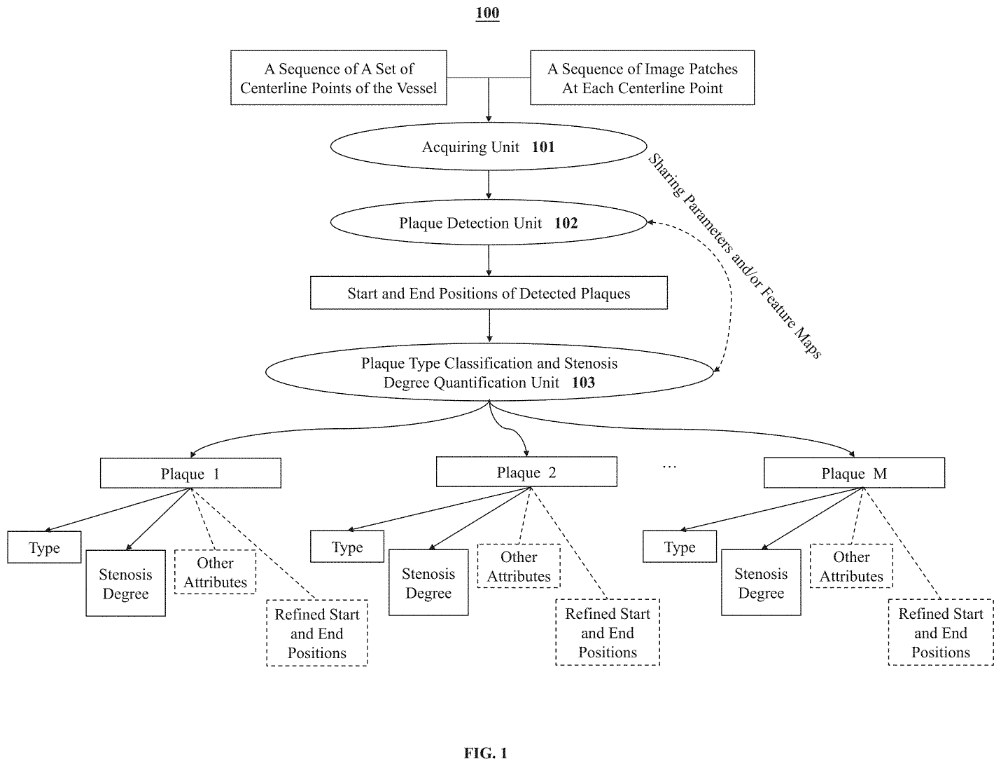

FIG. 1 shows a schematic diagram of a device 100 for vessel plaque analysis and its working principle according to an embodiment of the present disclosure. As shown in FIG. 1, the device 100 includes an acquiring unit 101, which can be configured to acquire a sequence ζ={ζ₁, ζ₂, . . . , ζ_{N}} of a set of centerline points of a vessel and a sequence X={X₁, X₂, . . . , X_{N}} of image patches at the respective centerline points, wherein N is the number of centerline points. In the present disclosure, the determination of the sequence of centerline points and the corresponding image patches can be performed without additional annotations, such as a segmentation mask of the vessel wall.

Various methods can be used to obtain the sequence of centerline points of a vessel and the sequence of image patches. For example, the processing software or workstation equipped for some medical imaging devices may already incorporate 3D reconstruction function and centerline extraction function, and thus may directly obtain the sequence of centerline points and extract the sequence of image patches at those centerline points based on the 3D model reconstructed by 3D reconstruction unit. For another example, the acquiring unit 101 may also be configured to first reconstruct a 3D model of the vessel based on a set of images along the vessel acquired by the medical imaging device, and extract the centerline before extracting the centerline points and the corresponding image patches. Taking the coronary artery as an example, the coronary CTA (CCTA) device is a commonly used non-invasive imaging device, which can perform reconstruction based on a series of CTA images along the extension direction of the coronary artery, and extract the centerline and image patches at corresponding centerline points. By using the CTA images of vessel, especially the existing reconstruction and centerline extraction functions in the processing software or workstation therefor, the device 100 for vessel plaque analysis can conveniently and quickly obtain the required input information without increasing the doctor's routine work flow, thus maintaining a low cost and making it highly user-friendly.

The device 100 may further include a plaque detection unit 102 and a plaque type classification and stenosis degree quantification unit 103. The plaque detection unit 102 may be configured to detect plaques and determine the start and end positions of each detected plaque, e.g., P={(p₁^{s},p₁^{e}), (p₂^{s},p₂^{e}), . . . , (p_{M}^{s}, p_{M}^{e})} using a first learning network based on the sequence of image patches at the centerline points, where M is the number of the detected plaques. In some embodiments, the first learning network may include an encoder and a plaque range generator connected sequentially. The encoder is configured to extract feature maps based on the sequence of image patches, and the plaque range generator is configured to generate the start position and the end position of each plaque based on the extracted feature maps. The start position and the end position of each detected plaque, e.g., P={(p₁^{s}, p₁^{e}), (p₂^{s}, p₂^{e}), . . . , (p_{M}^{s}, p_{M}^{e})} may be fed to the plaque type classification and stenosis degree quantification unit 103, which may be configured to, for each detected plaque 1, 2 . . . , M (M is integer less than or equal to N): determine the type of each plaques C={c_{ρ1}, c_{ρ2}, . . . , c_{ρM}} and the stenosis degree σ={σ_{ρ1}, σ_{ρ2}, . . . , σ_{ρM}} of the plaque by a second learning network reusing (sharing) at least part of the parameters of the first learning network and the extracted feature maps. In some embodiments, the type C of a plaque can be calcified, non-calcified and their combination. According to the application scenario, σ can be a quantitative number indicating the severity of the stenosis, a binary label (with or without stenosis), or a multi-categorial label. For example, according to the severity of stenosis, σ can fall into one or four categories: [0, 25%), [25%, 50%), [50, 75%), [75%, 100%]; or two categories [0, 50%), [50%, 100%]; or other number of categories.

By detecting physical attributes of the plaques (e.g., the positions of the plaques in an anatomical structure, the length of each plaque, the number of plaques), the type of the detected plaque (e.g., calcified, non-calcified and mixed) and the stenosis degree (e.g., stenosis level), complete, intuitive and comprehensive quantitative evaluation results may be provided for radiologists and cardiovascular experts, enabling them to make accurate diagnosis and significantly reduce the workload. Through reusing (sharing) of at least part of the parameters and feature maps between the second learning network and the first learning network in the detection phase and the training phase, the computational complexity can be effectively reduced and the processing time can be significantly shortened, which is particularly beneficial in medical image processing.

In some embodiments, the plaque type classification and stenosis degree quantification unit 103 may be further configured to determine other attributes of the plaque for each detected plaque M. Examples of such attributes may include, but not limited to, the related parameter of at least one of positive reconstruction, vulnerability, and napkin ring sign (presence or absence, severity level, etc.). These other attributes may assist in the diagnosis of certain types of vascular diseases, so that radiologists and cardiovascular experts can obtain more detailed reference information on demand. The availability of these additional attributes may further reduce their workload and increase the accuracy of diagnosis. In some embodiments, the start and end positions of the plaques may be further refined based on the local intermediate information (including but not limited to the probability related parameters of whether there exists a plaque, feature map, etc.) of each centerline point where the plaque M is located. Through the refinement based on the local distribution information, the non-plaque area (usually at the edge of the plaque) that is misidentified as part of the plaque can be eliminated from the plaque M, thereby sharpening the plaque edge to make its size more precise.

In some embodiments, the image patch may be a 2D image patch or a 3D image patch. Consistent with this disclosure, a 3D image patch may be a volume patch around each centerline point, or it may refer to a stack of 2D slice image patches around each centerline point along the centerline. For example, the 2D image patches may be orthogonal to the center line at the corresponding centerline point, but the orientation of the 2D image patches is not limited to this, and may also be inclined relative to the centerline. In some embodiments, the input of the first learning network has multiple channels, which are formed by resizing a set of image patches of multiple sizes at respective center points to be the same size and stacking the same (stacked into the multiple channels). By using a set of image patches of different sizes at the respective centerline points and later resizing them, the disclosed method avoids having to select a single size of image patches, which may usually have adverse effect on the result. For example, selecting a too large size will mix the image information of the surrounding tissue therein while selecting a too small size will risk losing certain image information of the vessel. Indeed, as the vessel changes its diameter at different positions, the appropriate size of the corresponding image patch may also be different consequently. By comparing the analysis results (such as plaque position, plaque type, and plaque stenosis degree) of multiple channels and determining the final analysis result through, for example, a majority decision strategy, the accuracy of the analysis results can be further improved.

The encoder may adopt learning networks of various architectures, such as but not limited to a multi-layer convolutional neural network, in which each layer may include a convolutional layer and a pooling layer, so as to extract feature maps from the input images for feeding to the subsequent processing stages. In some embodiments, the dimension of the convolution kernel of the convolution layer may be set to be the same as the dimension of the image patch.

For example, for 2D image patch, the encoder may use a set of 2D convolutional layer(s) and pooling layer(s) to generate corresponding feature maps for the 2D image patches at the respective centerline points. In some embodiments, conventional CNN architectures can be used directly, such as VGG (including multiple 3×3 convolutional layers and 2×2 max pooling layers) and ResNet (which adds skip connections between convolutional layers). In some other embodiments, a customized CNN architecture may also be used.

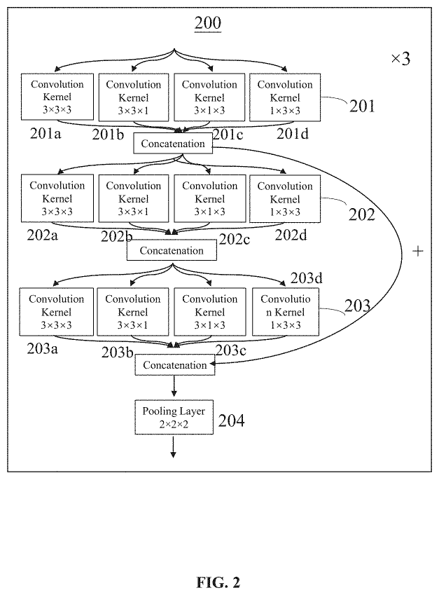

For 3D image patches, the encoder may use a combination of 3D convolutional layer(s) and pooling layer(s) to generate feature maps. In some embodiments, existing 3D CNN architectures may be used, such as 3D VGG and 3D ResNet. Alternatively, a customized 3D CNN architecture may also be used. In some embodiments, the encoder 200 may sequentially include multiple 3D convolutional layers and pooling layer(s), e.g., three convolutional layers 201, 202, and 203 and one pooling layer 204 as shown by FIG. 2. In some embodiments, each 3D convolution layer may include multiple 3D convolution kernels, which are configured to respectively extract feature maps in stereotactic space and each coordinate plane, and the feature maps extracted by each 3D convolution kernel may be concatenated and then fed to the next layer, thereby extracting information in different dimensions. Compared with only using the 3D convolution kernel (for example, the convolution kernel of 3×3×3) for extracting the feature maps in the stereotactic space, the local information in each coordinate plane may be maintained comprehensively and independently. For example, in case that the important information of the image patch is concentrated in a certain coordinate plane, if only the 3D convolution kernel for extracting the feature maps in the stereotactic space is used, such important information may be easily weakened or contaminated by information within other coordinate plane or information within stereotactic space. By the disclosed convolution kernels, not only can the local information in each coordinate plane be comprehensively and independently retained, but also the information distribution in the space can be taken into account, thereby obtaining more accurate analysis results.

In FIG. 2, as an example, each convolutional layer 201 (202 or 203) may include 4 3D convolution kernels 201a, 201b, 201c, 201d (202a, 202b, 202c, 202d, or 203a, 203b, 203c, 203d). In some embodiments, in order to extract the feature maps in the stereotactic space and each coordinate plane, a single 3D convolution kernel is respectively determined for each of the stereotactic space and three coordinate planes. In some alternative embodiments, multiple 3D convolution kernels may be determined for each of the stereotactic space and three coordinate planes.

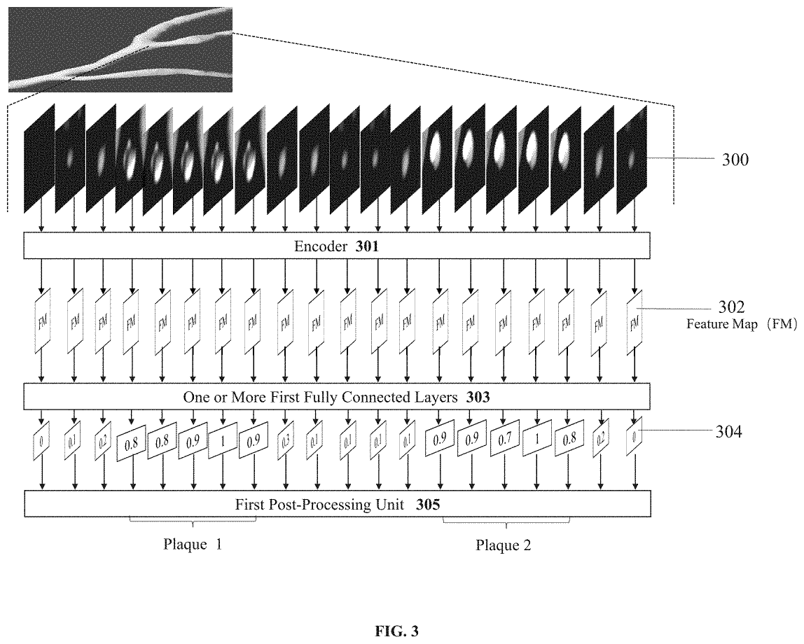

The plaque range generator can be implemented in various manners. The various embodiments are described in detail below with reference to FIGS. 3, 4 and 5 respectively.

FIG. 3 shows an embodiment of a plaque detection unit for a 2D image patch. The plaque detection unit may include an encoder 301, one or more first fully connected layers 303, and a first post-processing unit 305. In some embodiments, the one or more first fully connected layers 303 together with the first post-processing unit 305 constitute the plaque range generator. The encoder 301 may be configured to: extract the feature maps 302 at the 2D image patch level, based on a sequence of centerline points of vessel (20 centerline points in sequence are shown as an example in FIG. 2) and a sequence of 2D image patches 300 at each centerline point. A feature map 302 is extracted for each centerline point. These feature maps 302 are fed to the one or more first fully connected layers 303, which are configured to independently determine the probability related parameter 304 of existence of plaque in the 2D image patch at each centerline point based on the extracted feature maps 302. As shown in FIG. 3, the probability related parameters 304 of existence of plaque in the 2D image patches at this set of 20 centerline points are sequentially (0, 0.1, 0.2, 0.8, 0.8, 0.9, 1, 0.9, 0.3, 0.1, 0.1, 0.1, 0.1, 0.9, 0.9, 0.7, 1, 0.8, 0.2, 0). FIG. 3 shows an example in which probability serves as the probability related parameter 304, but the probability related parameter 304 may also be a parameter indicative of the probability in other forms, such as a score, etc. The first post-processing unit 305 may be configured to determine the centerline point where each plaque exists based on the probability related parameter 304 of existence of plaque. For example, each probability related parameters 304 may be compared with a certain threshold (e.g. 0.6), and when the threshold is exceeded, the corresponding centerline point may be considered to have a plaque existing thereon. The first post-processing unit 305 may also be configured to combine a set of consecutive centerline points that are determined to include a portion of a plaque to detect a complete plaque. For example, the 4th-8th centerline points with the probability related parameters 304 as (0.8, 0.8, 0.9, 1, 0.9) in order may be combined to detect plaque 1, and the first centerline point (for example, the fourth centerline point) and the last centerline point (for example, the eighth centerline point) in this set of centerline points may be determined as the start position p₁^{s} and end position p₁^{e} of the plaque 1.

For the plaque range generator based on the fully connected layers as shown in FIG. 3, the plaque type classification and stenosis degree quantification unit (not shown) based on fully connected layers may be used accordingly. Specifically, the second learning network may include one or more second fully connected layers (not shown), which are configured to reuse the feature maps 302 extracted by the encoder 301 for the 2D image patches at the centerline points determined to have plaque existing thereon as input. For example, for the detected plaque 1, the feature maps 302 extracted at the 4th-8th centerline points may be reused as input to the one or more second fully connected layers, so as to determine the type and stenosis degree of plaque 1. In some embodiments, it may further include a plaque instance refinement unit based on fully connected layer(s), which may be configured to, for each detected plaque: refine the start and end positions of the plaque by using one or more sixth fully connected layers based on the feature maps extracted by the encoder for the 2D image patches at the centerline points each determined to include a portion of a plaque. For example, for the detected plaque 1, the feature maps 302 extracted at the 4-8th centerline points may be reused as input to the one or more sixth fully connected layers, so as to determine the refined start and end positions of plaque 1. In some embodiments, in order to solve the problem of the different lengths of the detected plaques, pooling methods such as max pooling, adaptive pooling, and spatial pyramid pooling may be applied to the feature maps to generate pooled feature maps of the same size.

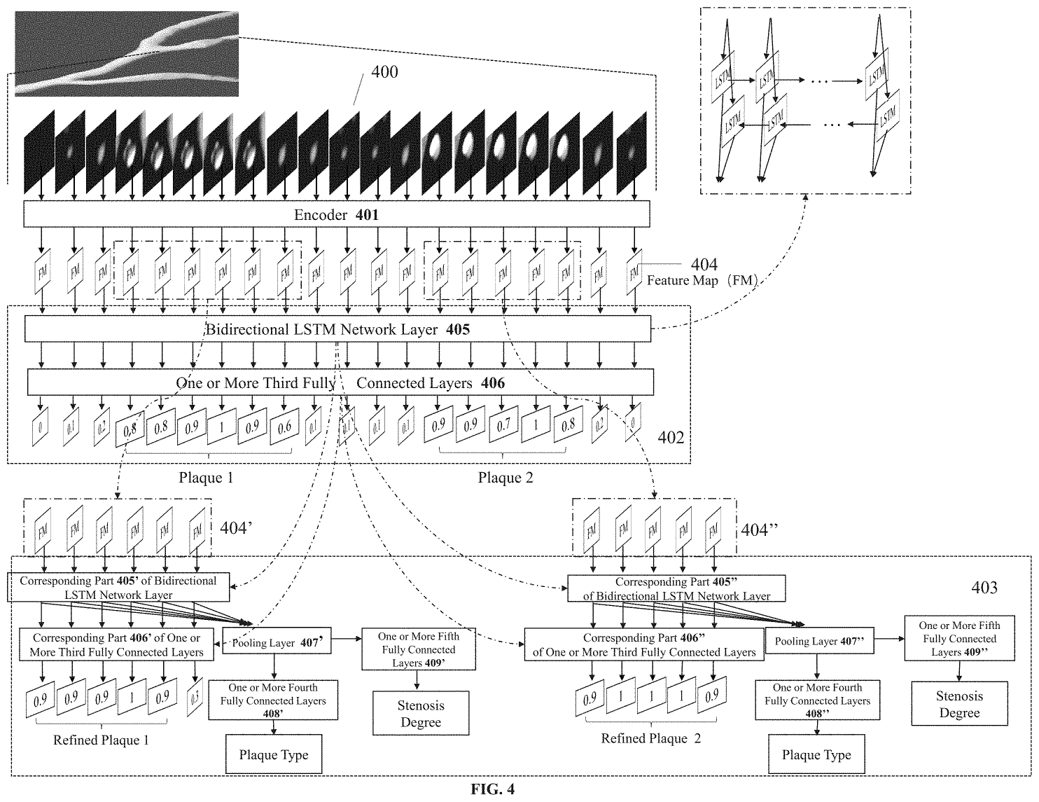

FIG. 4 shows an exemplary illustration of a learning network for vessel plaque analysis according to an embodiment of the present disclosure. The device may include an acquisition unit (not shown), a plaque detection unit (including an encoder 401 and a plaque range generator 402) and a plaque type classification and stenosis degree quantification unit 403. A sequence of centerline points of a vessel and a sequence of image patches 400 at the respective centerline points may be acquired. In case that the image patches are 2D image patches, the encoder 401 may be configured to extract feature maps 404 at the 2D image patch level. FIG. 4 shows a bidirectional LSTM network layer 405 is as an example of the learning network. It is contemplated that the bidirectional LSTM network layer can also be replaced with other types of RNN layer or convolutional RNN layer, such as but not limited to unidirectional LSTM, Bidirectional GRU, convolutional RNN, convolutional GRU, etc.

As shown in FIG. 4, the plaque range generator 402 sequentially includes a first recurrent neural network (RNN) or convolutional RNN layer (for example, a bidirectional LSTM network layer 405), one or more third fully connected layers 406, and second post-processing unit (not shown), wherein the first RNN or convolutional RNN layer together with one or more third fully connected layers 406 is configured to determine the probability related parameters (0, 0.1, 0.2, 0.8, 0.8, 0.9, 1, 0.9, 0.6, 0.1, 0.1, 0.1, 0.1, 0.9, 0.9, 0.7, 1, 0.8, 0.2, 0) of existence of plaque in the 2D image patches at the respective centerline point based on the extracted feature maps 404. The second post-processing unit is similar to the above-mentioned first post-processing unit 305, and therefore its description will not be repeated here. By including the first RNN or convolutional RNN layer, information can be aggregated across image patches along the center line, and context and sequential information may be taken into account. Further, by including the first convolutional RNN layer, it can also achieve acceleration on the GPU and preserve spatial information by replacing all element-wise operations with convolution operations.

Next, the plaque type classification and stenosis degree quantification unit 403 will be described in the context of the bidirectional LSTM network layer 405 and detected plaques 1 and 2. In some embodiments, parts 405′ and 405″ of the bidirectional LSTM network layer herein are examples of the second RNN or convolutional RNN layer, and the corresponding parts 406′ and 406″ of one or more third fully connected layers are examples of the one or more seventh fully connected layers in the present disclosure.

For the detected plaque 1, the feature maps 404′ extracted by the encoder 401 from the 2D image patches at the 4th-9th centerline points where the plaque 1 exists are reused as input. The input is fed to the pipeline for determining the type of plaque 1, which in turn includes the corresponding part 405′ (e.g., the sub-network in the bidirectional LSTM network layer 405 corresponding to 4-9th centerline points) of the bidirectional LSTM network layer, a pooling layer 407′, and one or more fourth fully connected layers 408′ to determine the plaque type.

For the detected plaque 2, the feature maps 404″ extracted by the encoder 401 for the 2D image patches at the 14th-18th centerline points where the plaque exists are reused as input. The pipeline for determining the type of detected plaque 2 is similar, including the corresponding part 405″ of the bidirectional LSTM network layer, the pooling layer 407″, and one or more fourth fully connected layer 408″, which are not repeated here.

The input is also fed to the pipeline for determining the stenosis degree of the plaque. For example, for plaque 1, the pipeline for determining the stenosis degree includes the corresponding part 405′ (e.g., the sub-network in the bidirectional LSTM network layer 405 corresponding to 4-9th centerline points) of the bidirectional LSTM network layer, the pooling layer 407′, and one or more fifth fully connected layer 409′. The pipeline for determining the stenosis degree of detected plaque 2 is similar, including the corresponding part 405″ (e.g., the sub-network in the bidirectional LSTM network layer 405 corresponding to 14-18th centerline points) of the bidirectional LSTM network layer, a pooling layer 407″, and one or more fifth fully connected layers 409″ to determine the steno sis degree of plaque 2.

In some embodiments, the device for vessel plaque analysis may further include a plaque instance refinement unit. FIG. 4 shows the plaque instance refinement unit as a constituent part of the plaque type classification and stenosis degree quantification unit 403. This is only an example, and the former may also be a unit independent of the latter. The composition of the plaque instance refinement unit will be described below by taking the detected plaque 1 as an example.

For the detected plaque 1, based on the feature maps 404′ extracted by the encoder 401 for the 2D image patches at the 4-9th centerline points determined to have the plaque 1 existing thereon, the corresponding part 405′ (e.g., the sub-network in the bidirectional LSTM network layer 405 corresponding to 4-9th centerline points) of the bidirectional LSTM network layer and the corresponding part 406′ of one or more third fully connected layers are used to refine plaque 1. In some embodiments, the start and end positions of the plaque may be refined. The structure of the above plaque instance refinement unit for plaque 1 is also applicable to other detected plaques. For example, for the detected plaque 2, based on the feature maps 404″ extracted by the encoder 401 for the 2D image patches at the 14-18th centerline points determined to have the plaque 2 existing thereon, the corresponding part 405″ (e.g., the sub-network in the bidirectional LSTM network layer 405 corresponding to 14-18th centerline points) of the bidirectional LSTM network layer and the corresponding part 406″ of one or more third fully connected layers are used to refine plaque 2. Specifically, corresponding part 406′ of one or more third fully connected layers may be used to respectively obtain the probability-related parameters after local enhancement processing at the centerline points where the plaque exists. For example, by comparing each probability-related parameter with a threshold, the centerline point at the edge that is more likely to belong to a non-plaque part (for example, the 9th centerline point for plaque 1) may be eliminated, so as to further determine the start and end positions of the refined plaque.

Other approaches may also be used to determine the start and end positions of each plaque. As an example, anchor-based generation approach may be used, and these anchors are then classified depending on whether there exists plaque or not. One exemplary strategy for generating anchors is to choose any pair of centerline points with a length larger than a threshold as a candidate and classify the plaque status. After the anchors are chosen, non-maximum suppression may be applied to combine candidate regions and obtain the start and end positions of each plaque.

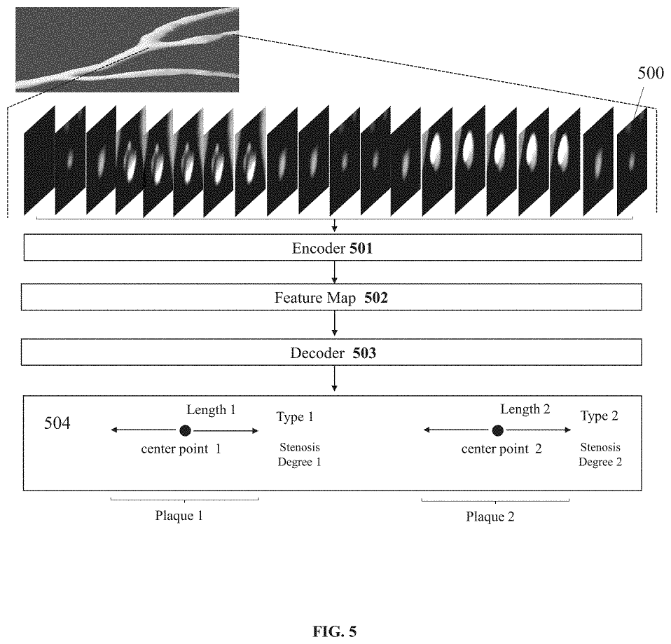

FIG. 5 shows an exemplary illustration of another learning network for vessel plaque analysis according to an embodiment of the present disclosure, wherein the plaque detection unit may be configured to detect plaques and determine the start and end positions of each detected plaque based on the sequence of the image patches 500 at the respective centerline points using a first learning network, which includes an encoder 501 and a plaque range generator in sequence. As shown in FIG. 5, the plaque range generator may be implemented as a combination of a decoder 503 and a third post-processing unit 504. The encoder 501 may be configured to determine the feature maps 502 based on the sequence of the image patches 500 at the respective centerline points. Note that although it is not particularly identified in FIG. 5, even if the image patch is a 3D image patch, the plaque may be identified with one-dimensional coordinates, whose the coordinate direction (taking the z coordinate as an example) may be along the centerline. The feature maps 502 may include multiple feature maps of different sizes and/or fields of view (different resolutions), and each feature map may be fed to different locations in the network structure of the decoder 503. The decoder 503 may be an upsampling path, which may recover the feature maps 502 to the original resolution of the image patch by combining the information of low-resolution features and high-resolution features.

The decoder 503 may be configured to output a two-element tuple (ρᵢ, Lᵢ) for each centerline point i, where i is the index number of the centerline point, ρᵢ is probability related parameter (such as but not limited to the score) of the plaque whose center point is located at the centerline point i, and Lᵢ is the associated plaque length. The following uses the score as an example of probability related parameter for description, but it should be noted that the probability related parameter is not limited to this. The third post-processing unit 504 may be configured to select a centerline point whose score reaches a threshold as the center of the plaque (for example, center point 1 and center point 2 are used as the center points of plaques 1 and 2 respectively). Based on the position p of the centerline point and the associated plaque length L (for example, length 1 and length 2), the start position p^{s} and end position p^{e} of the plaque may be respectively calculated as (p^{s}, p^{e})=(p−L/2, p+L/2). As an example, for plaque 1, its start position may be calculated as the position of center point 1−½*length of plaque 1 and the end position can be calculated as the position of the center point 1+½*length of plaque 1.

In some embodiments, the decoder 503 may be reused as or partially shared by the plaque type classification and stenosis degree quantification unit. For example, the decoder may be further configured to determine the plaque type C, and stenosis degree σᵢ of each centerline point i, for example, type 1 and stenosis degree 1 of plaque 1, and type 2 and stenosis degree 2 of plaque 2. In this case, the decoder 503 will output a four-element tuple (ρᵢ, Lᵢ, cᵢ, σᵢ) for each centerline point i that includes the probability of the centerline point i being the center of the plaque, the associated plaque length, the type and stenosis degree of the plaque. As a result, the plaque range generator and the plaque type classification and stenosis degree quantification unit may be integrated into a single unit.

In some embodiments, the decoder 503 may be further configured to also serve as a plaque instance refinement unit to refine the start position and end position for each detected plaque. In some embodiments, the decoder 503 may be further configured to determine, for each detected plaque, other attributes, such as but not limited to related parameters of at least one of positive reconstruction, vulnerability, and napkin ring sign.

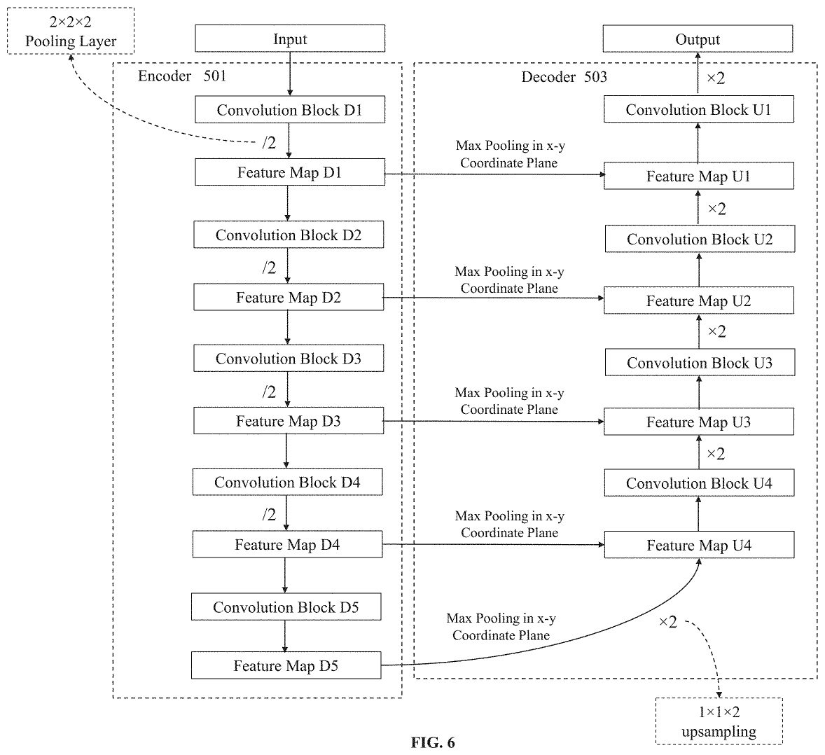

FIG. 6 shows an exemplary illustration of the encoder 501 and the decoder 503 in the learning network shown in FIG. 5, for 3D image patches. As shown in FIG. 6, both the encoder 501 and the decoder 503 are implemented by a fully convolutional neural network including multiple convolutional blocks. The convolution block Dn represents the nth downsampling convolution block, the feature map Dn represents the feature map obtained by the nth downsampling convolution block, the convolution block Un represents the nth upsampling convolution block, and the feature map Un represents the feature map used as the input of the nth upsampling convolution block. In FIG. 6, “/2” represents a pooling operation using a 2×2×2 pooling layer, and “×2” represents an upsampling operation using a 1×1×2 upsampling unit (note that the z coordinate is used as an example for plaque coordinates in FIG. 6, but other coordinates can also be used). In this manner, the plaque detection unit and the plaque type classification and stenosis degree quantification unit may be embedded in the same decoder 503, and a four element tuple (ρᵢ, Lᵢ, cᵢ, σᵢ) may be directly output for each centerline point i through the multi-channel output of the network. The four element tuple provides the probability of the centerline point i (i corresponding to the z coordinate) being the center of the plaque, and the length, type, and stenosis degree of the corresponding plaque assuming that centerline point i is the center of the plaque. In this manner, the architecture of the learning network is significantly simplified, and almost all feature maps and learning network parameters may be shared between the two units. Therefore, the workload and processing time in the training phase and the prediction phase may be further significantly reduced. In some embodiments, in addition to plaque type and stenosis degree, if needed, other attributes, such as positive reconstruction, vulnerability, and napkin ring sign related parameters, can also be analyzed in types.

As shown in FIG. 6, feature map D1, feature map D2, feature map D3, and feature map D4 extracted by each convolution block of the encoder 501, such as the convolution block D1, the convolution block D2, the convolution block D3, and the convolution block D4, may be individually pooled within a coordinate plane (such as x-y coordinate plane) perpendicular to the coordinate direction of the plaque (such as z direction). The pooled feature maps may be then fed to respective convolution blocks of the decoder 503 (e.g., convolution block U1, convolution block U2, convolution block U3, and convolution block U4). In some embodiments, the pooling used may include, but not limited to max pooling, average pooling, adaptive pooling, and spatial pyramid pooling, etc. Pooling of feature maps D1-D4 may use the same or different pooling methods. The resulted feature maps, that is, feature map U1, feature map U2, feature map U3, and feature map U4, may have different resolutions.

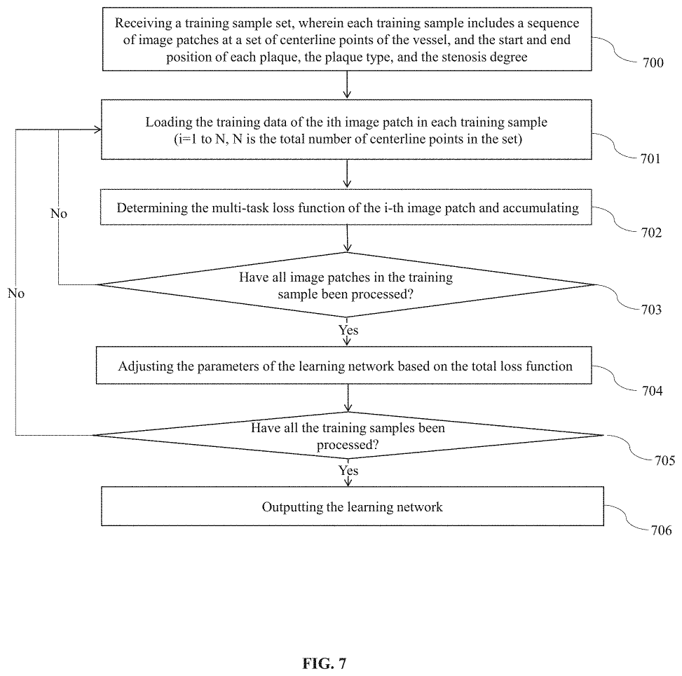

FIG. 7 shows a schematic flowchart for training a learning network for vessel plaque analysis according to an embodiment of the present disclosure. The process starts at step 700 for receiving a training sample set. Each training sample may include a sequence of image patches at a set of centerline points of the vessel, and the ground truth information of each plaque in that portion of the vessel, including, e.g., the start and end positions of each plaque, the plaque type label, and the stenosis degree label. Although FIG. 7 shows a process for training a learning network for detecting plaques and their start and end positions, plaque type, and stenosis degree as an example, it is contemplated that the process can be adapted by a person of ordinary skill in the art to train learning networks for detecting other attributes.

In step 701, each training sample may be loaded, specifically the training data of each image patch (the ith image patch, i=1 to N, N is the total number of centerline points in the set). In step 702, the multi-task loss function of the i-th image patch may be determined and then accumulated. In case that it is determined that all image patches in the training sample have been processed (Yes in step 703), the total loss function of the training sample may be obtained, and based on this, various optimization methods, such as but not limited to stochastic gradient descent method, RMSProp method or Adam method, may be used to adjust the parameters of the learning network (step 704). In this manner, training may be performed for each training sample until the training is completed for all the training samples in the training sample set, thereby obtaining and outputting the learning network (step 705). In some embodiments, the above described training process can be modified, e.g., to adopt minimum batch gradient descent, etc., to improve training efficiency.

The first learning network (used by the plaque detection unit) and the second learning network (used by the plaque type classification and stenosis degree quantification unit) in the learning network share at least part of the network parameters and the extracted feature maps. In step 702 of the training process, the loss function of the corresponding task of the first learning network may be calculated first, and the shared feature maps in the intermediate feature maps in the calculation process may be directly used to calculate the loss function of the corresponding task of the second learning network, thereby significantly reducing the computational cost of the multi-task loss function. In step 704 of the training process, after adjusting the parameters of the first learning network, the second learning network may adaptively adopt the adjusted shared parameters. For example, if a parameter of the first learning network is adjusted, the parameter, if shared with the second learning network, will be automatically adjusted in the second learning network. As a result, it further computational cost of parameter adjustment.

Embodiments of the corresponding multi-task loss function will be described below under various learning networks according to the present disclosure.

In an example, the multi-task loss function may be defined by the following formula:

[symbol]

=l_{d}+λ_{c}l_{c}+λ_{σ}l_{σ}++λ_{dr}l_{dr}+Σₖλₒₐₖlₒₐₖ, (1)

Wherein l_{d} refers to the plaque detection loss, l_{c} refers to the plaque classification loss, l_{σ} refers to the stenosis degree loss, l_{dr} refers to the detection refinement loss, and lₒₐₖ refers to the loss of other attributes (k refers to the serial number of other attributes). These attributes may be positive reconstruction, vulnerability and napkin ring sign, and λ_{c}, λ_{σ}, λ_{dr} and λₒₐₖ are weights associated with the respective losses.

When neither detection of other attributes nor refinement is needed, the corresponding items may be removed, and the multi-task loss function may be simplified as formula (2):

[symbol]

=l_{d}+λ_{c}l_{c}+λ_{σ}l_{σ}, (2)

The detailed loss function expressions are given below for two exemplary implementations of the learning network presented in FIG. 4 and FIG. 5.

For the 2D implementation as shown in FIG. 4, the respective components of the multi-task loss function may be defined as follows.

$${ℓ}_{d} = - \frac{1}{N} {\sum }_{i = 1}^{N} ( {γ}_{i} log {p}_{i} + ( 1 - {γ}_{i} ) log ( 1 - {p}_{i} ) ) , \qquad ( 3 )$$

l_{d} is the binary cross entropy loss, wherein pᵢ is the probability of the existence of plaque on the ith 2D image patch, γᵢ is the plaque status label (0 or 1) of the ith 2D image patch, and N is the total number of 2D image patches in the sequence.

$${ℓ}_{c} = - \frac{1}{CN} {\sum }_{j = 1}^{C} {\sum }_{i = 1}^{N} {γ}_{ij} log {p}_{ij} , \qquad ( 4 )$$

l_{c} is the multi-class cross entropy loss, wherein pᵢⱼ is the probability of the existence of the plaque of type j on the ith 2D image patch, γᵢⱼ is the one-hot plaque type label of the ith 2D image patch, and C=3 for the three plaque types. Depending on the application scenario, l_{σ} may be a cross entropy loss if the provided stenosis status is a binary (0 or 1) label or multi class label (such as different stenosis severity levels), or a L2 loss,

$${ℓ}_{σ} = \frac{1}{2 P} {\sum }_{i = 1}^{P} {( {σ}_{i} - {ζ}_{i} )}^{2} , \qquad ( 5 )$$

Where σᵢ is predicted stenosis score ranged from 0 to 1, ζᵢ is the stenosis ground truth for the i-th plaque, and P is the total number of detected plaques.

According to the parameter value type of other attributes (binary, multi-class, multi-label or continuous value), [symbol] ₒₐₖ may be cross entropy loss or Ln norm loss.

For the full convolution implementation of the learning network as shown in FIG. 5 and FIG. 6), the first learning network and the second learning network are actually integrated, and each component in the multi-task loss function may be refined as follows.

The plaque detection loss [symbol] _{d} may be calculated according to formula (6):

$${ℓ}_{d} = - \frac{1}{N} {\sum }_{i = 1}^{N} \{ \begin{matrix}{( 1 - {p}_{i} )}^{β} log {p}_{i} , {γ}_{i} = 1 \\ {( 1 - {γ}_{i} )}^{α} {p}_{i}^{β} log ( 1 - {p}_{i} ) , {γ}_{i} < 1\end{matrix} \qquad ( 6 )$$

where [symbol] _{d} is the plaque detection loss, p₁ is the probability related parameter (for example but not limited to a score) of the image patch at the ith centerline point with respect to the center of the plaque, γᵢ is the plaque center status label for the image patch at the ith centerline point after conversion (for example but not limited to Gaussian transformation), with values ranged from 0 to 1, N is the total number of centerline points, and α and β are constants. As an example, α may be set as α=2, and β may be set as β=4.

Various other components in the multi-task loss function, for example, [symbol] _{c}, [symbol] _{σ} and [symbol] ₒₐₖ, are losses described above. In an exemplary implementation of full convolution, refinement may not be detected, and [symbol] _{dr}=0 accordingly.

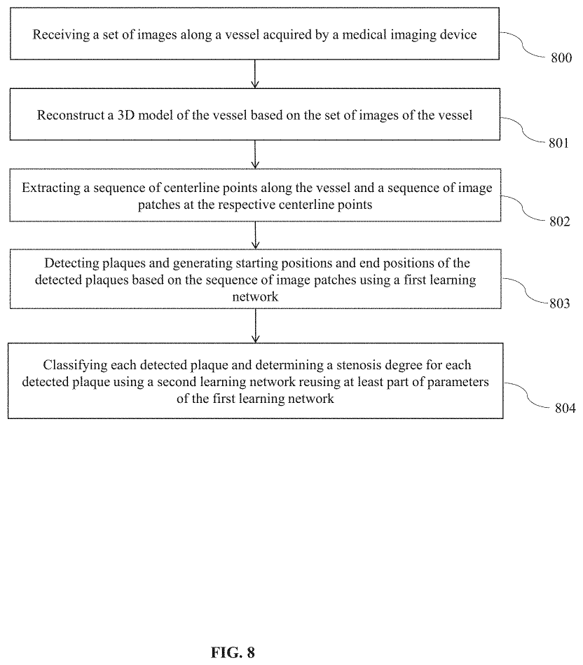

FIG. 8 shows a flowchart an exemplary method for vessel plaque analysis using a learning network, according to the embodiment of the present disclosure. Although FIG. 8 shows a process for detecting plaques and their start and end positions, plaque type, and stenosis degree as an example, it is contemplated that the process can be adapted by a person of ordinary skill in the art to detect other attributes associated with plaques.

In step 800, a set of images along a vessel acquired by a medical imaging device are received. For example, the images may be CTA images of the vessel acquired by a CTA device. In step 801, a 3D model of the vessel may be reconstructed based on the set of images of the vessel. In various embodiments, the reconstruction may be performed by the medical imaging device, the plaque analysis device, or another separate device in communication with the plaque analysis device. In step 802, a sequence of centerline points are extracted along the vessel, and a sequence of image patches are extracted at the respective centerline points. In some embodiments, the image patch at each centerline point is one of a 2D image patch orthogonal to the center line at the corresponding centerline point, a stack of 2D slice image patches along the centerline around the corresponding centerline point, or a 3D image patch around the corresponding centerline point.

In step 803, one or more plaques are detected and the starting position and end position of each detected plaque is generated based on the sequence of image patches using a first learning network. In some embodiments, the first learning network includes an encoder configured to extract feature maps based on the sequence of image patches and a plaque range generator configured to generate a start position and an end position of each plaque based on the extracted feature maps. For example, the first learning network can be the learning networks as shown in FIG. 3 or FIG. 4.

In some embodiments, when the image patch is 3D, the encoder of the first learning network may sequentially include multiple 3D convolutional layers and pooling layers, and each 3D convolution layer may include multiple 3D convolution kernels. Accordingly, in step 803, the encoder may respectively extract feature maps in stereotactic space and each coordinate plane, concatenate feature maps extracted by the 3D convolution kernels, and feed the concatenated feature map to the corresponding pooling layer.

In some embodiments, when the image patch is 2D, the encoder may be configured to extract the feature maps in 2D. The plaque range generator may include one or more first fully connected layers. Accordingly, in step 803, the one or more first fully connected layers may determine a probability related parameter of existence of a plaque in the 2D image patch at each centerline point based on the extracted feature maps, determine the centerline points associated with the existence of the plaque based on the probability related parameters, combining a set of consecutive centerline points associated with the existence of the plaque, and designate the first centerline point and the last centerline point in the set of consecutive centerline points as the start position and end position of the plaque.

In some embodiments, as part of step 803, the plaque range generator may further select a centerline point whose probability related parameter exceeds a threshold as a center of the plaque, determine a plaque length of the plaque, and determine the start position and the end position of the plaque based on the position of the selected centerline point and the plaque length.

In step 804, each detected plaque is classified (e.g., according to its type) using a second learning network reusing at least part of parameters of the first learning network. In some embodiments, as part of step 804, the second learning network can be used to further determine a stenosis degree for each detected plaque along with its type. In some embodiments, the second learning network may additionally reuse feature maps extracted by the first learning network. For example, the second learning network may include one or more fully connected layers that reuse the feature maps extracted by the encoder at the centerline points associated with the existence of the plaque.

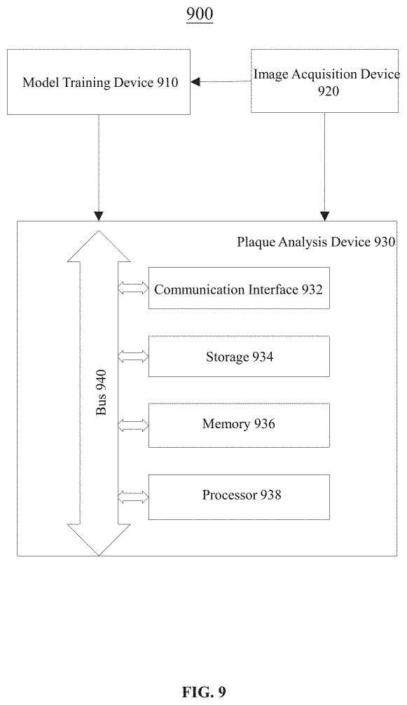

FIG. 9 shows a structural block diagram of a system 900 for vessel plaque analysis according to an embodiment of the present disclosure. In some embodiments, the system may include a model training device 910, an image acquisition device 920, and a plaque analysis device 930. In some embodiments, the system may only include a plaque analysis device 930, specifically including a communication interface 932 configured to acquire a set of images along the vessel acquired by the image acquisition device 910c(for example, a medical imaging device) and a processor 938. The processor 938 may be configured to: reconstruct a 3D model of the vessel based on a set of images of the vessel, and extract a sequence of centerline points of the vessel and a sequence of the image patches at the respective centerline points. The processor 938 may be further configured to extract feature maps based on the sequence of image patches using a first learning network, and generate start position and end position of each plaque based on the extracted feature maps. The processor 938 may be further configured to determine the type and stenosis degree for each detected plaque, by a second learning network reusing at least part of the parameters of the first learning network and the extracted feature maps. If necessary, the processor 938 may also be further configured to determine other attributes of each plaque, such as related parameter of at least one of positive reconstruction, vulnerability, and napkin ring sign; and may also be further configured to refine the start position and end position of each plaque. Various embodiments of the first learning network and the second learning network are described above, which will not be repeated here. The hardware structure of the plaque analysis device will be described in detail below, and the hardware structure can also be applied to the model training device 910, which will not be repeated here.

In some embodiments, the vessel may include any one of coronary artery, carotid artery, abdominal aorta, cerebral vessel, ocular vessel, and femoral artery, and the image acquisition device 920 may include but is not limited to a CTA device. Specifically, the image acquisition device 920 may include CT, MRI, and an imaging device including any one of functional magnetic resonance imaging (such as fMRI, DCE-MRI, and diffusion MRI), cone beam computed tomography (CBCT), positron emission tomography (PET), Single-photon emission computed tomography (SPECT), X-ray imaging, optical tomography, fluorescence imaging, ultrasound imaging and radiotherapy field imaging, etc.

In some embodiments, the model training device 910 may be configured to train learning networks (for example, the first learning network and the second learning network), and transmit the trained learning network to the plaque analysis device 930, and the plaque analysis device 930 may be configured to perform plaque analysis for the vessel based on the sequence of centerline points of the vessel and the sequence of image patches at respective centerline points by using the trained learning network. In some embodiments, the model training device 910 and the plaque analysis device 8930 may be integrated in the same computer or processor.

In some embodiments, the plaque analysis device 930 may be a special purpose computer or a general-purpose computer. For example, the plaque analysis device 930 may be a computer customized for a hospital to perform image acquisition and image processing tasks, or may be a server in the cloud. As shown in the figure, the plaque analysis device 930 may include a communication interface 932, a processor 938, a memory 936, a storage 934, and a bus 840, and may also include a display (not shown). The communication interface 932, the processor 938, the memory 936, and the storage 934 may be connected to the bus 940 and may communicate with each other through the bus 940.

In some embodiments, the communication interface 932 may include a network adapter, a cable connector, a serial connector, a USB connector, a parallel connector, a high-speed data transmission adapter (such as optical fiber, USB 3.0, Thunderbolt interface, etc.), a wireless network adapter (Such as WiFi adapter), telecommunication (3G, 4G/LTE, 5G, etc.) adapters, etc. The plaque analysis device 930 may be connected to the model training device 910, the image acquisition device 920, and other components through the communication interface 932. In some embodiments, the communication interface 932 may be configured to receive a trained learning network from the model training device 910, and may also be configured to receive medical images from the image acquisition device 920, such as a set of images of vessels, specifically, for example, the two vessel CTA images of the vessel with proper projection angle and sufficient filling to realize 3D reconstruction of vessel, but not limited to this.

In some embodiments, the memory 936/storage 934 may be a non-transitory computer-readable medium, such as read only memory (ROM), random access memory (RAM), phase change random access memory (PRAM), static random access memory access memory (SRAM), dynamic random access memory (DRAM), electrically erasable programmable read-only memory (EEPROM), other types of random access memory (RAM), flash disks or other forms of flash memory, cache, register, static memory, compact disc read only memory (CD-ROM), digital versatile disk (DVD) or other optical memory, cassette tape or other magnetic storage devices, or any other possible non-transitory medium used to store information or instructions that can be accessed by computer equipment, etc. In some embodiments, computer-executable instructions are stored on the medium. The computer-executable instructions, when executed by the processor 806, may perform at least the following steps: obtaining a sequence of a set of centerline points of a vessel and a sequence of image patches at each centerline point; extracting feature maps based on the sequence of image patches at each centerline point using the first learning network, and generating the start position and end position of each plaque based on the extracted feature maps; and determining the type and stenosis degree for each detected plaque by the second learning network reusing at least part of the parameters of the first learning network and the extracted feature maps.

In some embodiments, the storage 934 may store a trained network and data, such as feature maps generated while executing a computer program. In some embodiments, the memory 936 may store computer-executable instructions, such as one or more image processing (such as plaque analysis) programs. In some embodiments, various disclosed processes can be implemented as applications on the storage 934, and these applications can be loaded to the memory 936, and then executed by the processor 938 to implement corresponding processing steps. In some embodiments, the image patches may be extracted at different granularities and stored in the storage 934. The feature maps can be read from the storage 934 and stored in the memory 936 one by one or simultaneously.

In some embodiments, the processor 938 may be a processing device including one or more general processing devices, such as a microprocessor, a central processing unit (CPU), a graphics processing unit (GPU), and so on. More specifically, the processor may be a complex instruction set computing (CISC) microprocessor, a reduced instruction set computing (RISC) microprocessor, a very long instruction word (VLIW) microprocessor, a processor running other instruction sets, or a processor that runs a combination of instruction sets. The processor may also be one or more dedicated processing devices, such as an application specific integrated circuit (ASIC), a field programmable gate array (FPGA), a digital signal processor (DSP), a system on a chip (SoC), etc. The processor 938 may be communicatively coupled to the memory 936 and configured to execute computer executable instructions stored thereon.

In some embodiments, the model training device 910 may be implemented using hardware specially programmed by software that executes the training process. For example, the model training device 910 may include a processor and a non-volatile computer readable medium similar to the plaque analysis device 930. The processor implements training by executing executable instructions for the training process stored in a computer-readable medium. The model training device 910 may also include input and output interfaces to communicate with the training database, network, and/or user interface. The user interface may be used to select training data sets, adjust one or more parameters in the training process, select or modify the framework of the learning network, and/or manually or semi-automatically provide prediction results associated with the image patch sequence for training (for example, marked ground truth).

Another aspect of the disclosure is directed to a non-transitory computer-readable medium storing instructions which, when executed, cause one or more processors to perform the methods, as discussed above. The computer-readable medium may include volatile or non-volatile, magnetic, semiconductor-based, tape-based, optical, removable, non-removable, or other types of computer-readable medium or computer-readable storage devices. For example, the computer-readable medium may be the storage device or the memory module having the computer instructions stored thereon, as disclosed. In some embodiments, the computer-readable medium may be a disc or a flash drive having the computer instructions stored thereon.

It is intended that the description and examples are to be regarded as exemplary only, with the true scope being indicated by the appended claims and their equivalents.

What is claimed is:

1. A method for vessel plaque analysis, comprising:
receiving a set of images along a vessel acquired by a medical imaging device;
determining a sequence of centerline points along the vessel and a sequence of image patches at the respective centerline points based on the set of images;
detecting plaques based on the sequence of image patches using a first learning network, wherein the first learning network includes an encoder configured to extract feature maps based on the sequence of image patches and a plaque range generator configured to generate a start position and an end position of each plaque based on the extracted feature maps; and
classifying each detected plaque and determining a stenosis degree for the detected plaque, using a second learning network reusing at least part of parameters of the first learning network and the extracted feature maps.

2. The method of claim 1, wherein the vessel includes any one of a coronary artery, a carotid artery, an abdominal aorta, a cerebral vessel, an ocular vessel, and a femoral artery, wherein the set of images received are CTA images of the vessel acquired by a computer tomography angiography (CTA) device.

3. The method of claim 1, wherein classifying each detected plaque further comprises determining parameters related to at least one of positive reconstruction, vulnerability, and napkin ring sign for each detected plaque.

4. The method of claim 1, wherein the image patch at each centerline point is one of a 2D image patch orthogonal to the center line at the corresponding centerline point, a stack of 2D slice image patches along the center line around the corresponding centerline point, or a 3D image patch around the corresponding centerline point.

5. The method of claim 1, wherein the encoder includes a convolutional layer and a pooling layer, wherein a convolution kernel of the convolution layer has a same dimension as the image patch.

6. The method of claim 5, wherein the first learning network has multiple input channels, wherein the method further comprises:
resizing a set of image patches of different sizes at respective center points to a same size; and
stacking the set of resized image patches into the multiple input channels.

7. The method of claim 1, wherein the image patch is a 3D image patch, wherein the encoder sequentially includes multiple 3D convolutional layers and pooling layers, wherein each 3D convolution layer includes multiple 3D convolution kernels,
wherein the method further comprises:
respectively extracting feature maps in stereotactic space and each coordinate plane;
concatenating feature maps extracted by the 3D convolution kernels; and
feeding the concatenated feature map to the corresponding pooling layer.

8. The method of claim 1, wherein the image patch is a 2D image patch, wherein the method further comprises:
determining a probability related parameter of existence of a plaque in the 2D image patch at each centerline point based on the extracted feature maps;
determining the centerline points associated with the existence of the plaque based on the probability related parameters;
combining a set of consecutive centerline points associated with the existence of the plaque; and
designating the first centerline point and the last centerline point in the set of consecutive centerline points as the start position and the end position of the plaque.

9. The method of claim 8, wherein the second learning network includes one or more fully connected layers that reuse the feature maps extracted by the encoder at the centerline points associated with the existence of the plaque.

10. The method of claim 8, wherein the probability related parameter is determined using a first recurrent neural network (RNN) or convolutional RNN layer, and one or more fully connected layers.

11. The method of claim 8, further comprising:
selecting a centerline point whose probability related parameter exceeds a threshold as a center of the plaque;
determining a plaque length of the plaque; and
determining the start position and the end position of the plaque based on the position of the selected centerline point and the plaque length.

12. The method of claim 8, further comprising:
refining the start and end positions of the plaque based on the feature maps extracted by the encoder for the 2D image patches at the centerline points associated with the existence of the plaque.

13. The method of claim 10, further comprises refining the start and end positions of the plaque, by using a second RNN or convolutional RNN layer and one or more fully connected layers reusing a sub-network in the one or more fully connected layers used to determine the probability related parameter.

14. The method of claim 1, wherein the first learning network and the second learning network are jointly trained using a multi-task loss function.

15. A system for vessel plaque analysis, wherein the system includes:
an interface configured to receive a set of images along a vessel acquired by a medical imaging device; and
a processor configured to:
reconstruct a 3D model of the vessel based on the set of images of the vessel;
extract a sequence of centerline points along the vessel and a sequence of image patches at the respective centerline points;
detect plaques based on feature maps extracted from the sequence of image patches and generate a start position and an end position of each plaque based on the extracted feature maps, using a first learning network; and
classify each detected plaque and determine a stenosis degree for each detected plaque, using a second learning network reusing at least part of parameters of the first learning network and the extracted feature maps.

16. The system of claim 15, wherein the vessel includes any one of a coronary artery, a carotid artery, an abdominal aorta, a cerebral vessel, an ocular vessel, and a femoral artery, and the medical imaging device includes a computer tomography angiography (CTA) device.

17. The system of claim 15, wherein the image patch is a 3D image patch, wherein the first learning network includes an encoder that sequentially includes multiple 3D convolutional layers and pooling layers, wherein each 3D convolution layer includes multiple 3D convolution kernels,
wherein the processor is further configured to:
respectively extract feature maps in stereotactic space and each coordinate plane;
concatenate feature maps extracted by the 3D convolution kernels; and
feed the concatenated feature map to the corresponding pooling layer.

18. The system of claim 15, wherein the image patch is a 2D image patch, wherein the processor is further configured to:
determine a probability related parameter of existence of a plaque in the 2D image patch at each centerline point based on the extracted feature maps;
determine the centerline points associated with the existence of the plaque based on the probability related parameters;
combine a set of consecutive centerline points associated with the existence of the plaque; and
designate the first centerline point and the last centerline point in the set of consecutive centerline points as the start position and the end position of the plaque.

19. The system of claim 15, wherein the first learning network and the second learning network are jointly trained using a multi-task loss function.

20. A non-transitory computer-readable storage medium having computer-executable instructions stored thereon, wherein the computer-executable instructions, when executed by a processor, perform a method for vessel plaque analysis, the method comprising:
receiving a set of images along a vessel acquired by a medical imaging device;
determining a sequence of centerline points along the vessel and a sequence of image patches at the respective centerline points based on the set of images;
detecting plaques based on the sequence of image patches using a first learning network, wherein the first learning network includes an encoder configured to extract feature maps based on the sequence of image patches and a plaque range generator configured to generate a start position and an end position of each plaque based on the extracted feature maps; and
classifying each detected plaque and determining a stenosis degree for the detected plaque, using a second learning network reusing at least part of parameters of the first learning network and the extracted feature maps.
